(19) United States
(12) Patent Application Publication
(10) Pub. No.: US 2016/0312005 A1
(43) Pub. Date: Oct. 27, 2016

(54) FATTY ACID DIAMIDES COMPRISING STEARIC HYDROXYACIDS AS ORGANOGELATORS

(71) Applicant: Arkema France, Colombes (FR)

(72) Inventors: Michael Y. Bernard, Enghien Les Bains (FR); Christophe Duquenne, Paris (FR); Guillaume P. Monnier, Avrigny (FR)

(21) Appl. No.: 14/392,016

(22) PCT Filed: Jul. 24, 2013
(86) PCT No.: PCT/FR2013/051785

§ 371 Date: Jul. 18, 2016

(30) Foreign Application Priority Data
Jul. 27, 2012 (FR) FR12.57299

(51) Int. Cl. C08K 5/20; C09D 7/12; C07C 235/06

(52) U.S. Cl. CPC C08K 5/20; C07C 235/06; C09D 7/1233

(57) ABSTRACT

The invention is directed to a fatty acid diamide comprising in its structure specific hydroxycarboxylic acids, and to the use of this product as an organogelator or rheology agent, also known as a rheology additive, in particular in coating, glue or adhesive, molding, mastic, sealing or cosmetic compositions.

The invention relates first to a fatty acid diamide comprising in its structure specific hydroxycarboxylic acids, and to the use of this product as an organogelator or rheology agent, also known as a rheology additive, in particular in coating, glue or adhesive, molding, mastic, sealing or cosmetic compositions.

Fatty acid amides and in particular diamides based on 12-hydroxystearic acid are already known as organogelators, i.e. small organic molecules that are capable of gelling all kinds of organic solvents even at relatively low mass concentrations (less than 1% by mass) or as rheology additives, i.e. additives for modifying the rheology of an application formulation. They make it possible to obtain, for example, a thixotropic or pseudoplastic effect.

U.S. Pat. No. 4,128,436 describes a rheology control agent which comprises a mixture in particulate form of hydrogenated castor oil with a polyamide oligomer based on hydroxystearic acid, α-ω saturated primary diamines and α-ω saturated dicarboxylic acids or hydrogenated acid dimers.

U.S. Pat. No. 3,977,894 describes a rheology additive for non-aqueous systems based on organic modified montmorillonite clays self-activated in that said clays are in the homogeneous mixture with two solid waxes, one based on 12-HSA triglyceride and the other based on an amide based on a primary diamine and 12-HSA.

US 2006/0 047 046 describes the use of a thixotropic agent for an anaerobic adhesive composition, this agent being based on a mineral agent modified with organic salts or based on an organic agent optionally combined with a mineral particulate agent that is nonmodified or modified with organic salts. Organic agents that are mentioned are hydrogenated castor oils or waxes or 12-HSA triglycerides, optionally in the presence of a C₁₀ to C₂₄ fatty acid amide so as to improve the dispersibility of the triglycerides.

FR 2 281 162 describes the solidification or gelation of nonpolar organic liquids such as hydrocarbons using as solidification or gelling agent esters, amides or amine salts of amino acids, which are all N-acylated.

EP 2 098 502 describes C₃ to C₂₀₀ alkylamide or diamide compounds comprising a C₅ to C₈ cyclic structure, these structures being obtained from a diamine or a diacid or an amino acid of cyclic C₅ to C₈ structure and, respectively, of at least two monoacids or two monoamines that are identical or different or of a monoacid and a monoamine in the case of an amino acid, of identical or different length, the monoamines or monoacids being C₃ to C₂₀₀. One among the extensive list of monoacids mentioned is 12-HSA. These alkylamides or these diamides are recommended for use in inkjet printing inks which afford improvements in terms of viscosity, homogeneity and cost.

V. A. Mallia et al. describe in Langmuir 2009, 25 (15) 8615-8625 nitrogenous derivatives such as amides or amines, of 12-HSA and of stearic acid and compare their organogelling properties in various solvents.

12-Hydroxystearic acid, also known as 12HSA or 12-HSA hereinbelow, is thus commonly used as a starting material for the preparation of fatty amides. However, this hydroxycarboxylic acid is derived from a route whose sole source is castor oil. On account of the rapid development of certain applications extensively using castor oil directly or in the form of its derivatives, its consumption has considerably increased, giving rise to problems of availability and of pressure on the prices of these starting materials derived from the castor oil route, such as 12-HSA. There is thus an increasing need to find an alternative solution to 12-HSA by seeking novel starting materials derived from a route independent of castor oil, which is both abundant and also of renewable (or biosourced) origin, which can partially replace, in particular at least 10% and preferably totally, i.e. 100%, 12-HSA, while at the same time maintaining very satisfactory organogelling or rheological performance qualities for various applications at weight contents of less than 5%, in particular not exceeding 1% for a coating application.

None of the cited prior art documents describes or suggests the solution of such a technical problem or describes or suggests the solution of the present invention.

The present invention is directed toward novel fatty diamides which have a reduced and preferably zero content of 12-hydroxystearic acid (12-HSA), which is obtained from ricinoleic oil as starting material and commonly used as a fatty diamide component. This is performed by partially replacing it, in particular at least 20% and preferably totally, i.e. 100%, with other saturated fatty hydroxycarboxylic acids having a source different from that of castor oil, this source possibly being that of oleic oil. Specifically, the hydroxylation of oleic acid gives access to 9-hydroxystearic and 10-hydroxystearic acids, abbreviated, respectively, as 9-HSA and 10-HSA. This replacement is directed in this manner toward having performance qualities of organogelator in organic solvent medium that are entirely satisfactory in terms of thixotropic power, in particular for weight contents of less than 5%, more particularly less than 1% by weight in the targeted application, and which may, in certain cases, be at least as good as those of the organogelators of the prior art, in particular based exclusively on 12-HSA. The partial or total replacement should therefore not affect the rheological performance qualities of the diamide products thus obtained, while at the same time respecting a durable environment with starting materials of renewable origin.

With this aim, the first subject of the present invention is directed generally toward a fatty acid diamide in which the acid 12-HSA is partially and more particularly preferably totally replaced with another saturated fatty carboxylic acid based on stearic acid bearing a hydroxyl group in position 9 or 10, of renewable origin, and the respective mixtures thereof.

Another subject of the invention is an additive, in particular a rheology additive, comprising a diamide of the invention, said additive existing or used in the form of a paste that is preactivated and preconcentrated in a polar organic solvent or plasticizer.

The invention also relates to an organogelator and more particularly to a rheology agent comprising said diamide and to the use of said diamide per se.

Finally, the invention covers an organic binder composition comprising as rheology agent at least one diamide according to the present invention.

Thus, the first subject of the invention is a fatty acid diamide which comprises at least one product of reaction of a mixture comprising or consisting of:
a) at least one diamine selected from:
   a linear aliphatic C₂ to C₁₂, preferably C₂ to C₈ and more preferentially C₂ to C₆ diamine, and/or
   a C₆ to C₁₈ and preferably C₆ to C₁₂ cycloaliphatic diamine, and/or
   an aromatic diamine preferably of C₆ to C₁₂, more preferentially xylylenediamine and/or phenylenediamine,
b) at least one saturated hydroxycarboxylic fatty acid selected from 9-hydroxystearic acid (9-HSA) and/or 10-hydroxystearic acid (10-HSA) in the presence or absence of 12-hydroxystearic acid (12-HSA),
c) optionally, at least one monoacid selected from saturated, non-hydroxylated C₆ to C₁₈, preferably C₆ to C₁₅ and more preferentially C₆ to C₁₂ linear carboxylic acids,
d) optionally, at least a second diamine different from a) and selected from linear aliphatic C₂ to C₁₂, preferably C₂ to C₈ and more preferentially C₂ to C₆ amines.

According to a more particular case of the invention, said 12-hydroxystearic acid (12-HSA) is present and the content of the other hydroxystearic acids, i.e. 9-hydroxystearic acid and/or 10-hydroxystearic acid, ranges from 10% to 99%, preferably from 20% to 99% and more preferentially from 30% to 99% by weight of component b).

More particularly, in said diamide of the invention, said component b) is a mixture of 9-hydroxystearic acid and/or of 10-hydroxystearic acid and of 12-hydroxystearic acid, with a mole ratio of 9-HSA and/or 10-HSA to 12-HSA ranging from 10/90 to 99/1, more preferentially from 20/80 to 99/1 and more particularly from 30/70 to 99/1.

According to another preferred option, in said diamide, 12-hydroxystearic acid is absent and is totally replaced, i.e. to 100%, with 9-hydroxystearic acid and/or 10-hydroxystearic acid. The replacement of 12-HSA, whether it is partial or total, is preferably performed with a mixture of 9-HSA and 10-HSA, as obtained without particular separation from oleic acid after hydroxylation of oleic acid, bearing an ethylenic unsaturation in position 9, which unsaturation is saturated by said hydroxylation with production of said mixture of 9-HSA and 10-HSA. This mixture may be considered as being equimolar.

According to a particular case, said diamide bears two amide functions based on the same hydroxy acid b) or based on two different hydroxy acids b).

According to another preferred option of the invention, said monoacid c) is present in a content such that the mole ratio of said hydroxy acid b) to said monoacid c) is from 1/2 to 4/1. Even more particularly, said diamide of the invention bears one amide function based on a hydroxy acid b) and another function based on said monoacid c), which means that the mole ratio b/c is 1/1.

In general, the amine/acid mole ratio may range from 0.9 to 1.1 and preferably corresponds to the stoichiometric ratio of 1/1.

According to another possibility, said diamide according to the invention comprises at least two and preferably at least three different reaction products, as defined above. Thus, said diamide may be a binary or ternary, quaternary, or more, mixture of different reaction products as described above. According to a more particular case, according to this possibility, said diamide of the invention may comprise a mixture of products comprising products having the following formulae:

b1-a1-c1

b1-a1-b1

b1-a1-b2

and preferably:

b1-a1-c1

b1-a1-b1

b1-aa-b2

b2-a1-c1

b2-a1-b2

with a1: diamine residue according to a), b1: 9-HSA residue, b2: 10-HSA residue and c1: non-hydroxylated monoacid residue according to monoacid c).

As examples of suitable linear aliphatic diamines that are preferred for the diamine component a) of said diamide, mention may be made of: ethylenediamine, propylenediamine, butylene(or tetramethylene)diamine, pentamethylenediamine, hexamethylenediamine and preferably ethylenediamine or hexamethylenediamine.

As examples of cycloaliphatic diamines that are still suitable according to the component a), mention may be made of: 1,3-, 1,4- and 1,2- and in particular 1,3- or 1,4-cyclohexanediamine, isophoronediamine, 1,3-, 1,4- or 1,2-bis(aminomethyl)cyclohexane (hydrogenation derivative of, respectively, m-, p-, o-xylylenediamine), preferably 1,3- or 1,4-bis(aminomethyl)cyclohexane, decahydronaphthalenediamine, bis(3-methyl)-bis(4-aminocyclohexyl)methane (BMACM) or bis(4-aminocyclohexyl)methane (BACM), 1-{[4-(aminomethyl)cyclohexyl]oxy}propan-2-amine. The preferred cycloaliphatic diamines are chosen from: 1,3- or 1,4-cyclohexanediamine, 1,3-, 1,4- or 1,2-bis(aminomethyl)cyclohexane, isophoronediamine, bis(4-aminocyclohexyl)methane.

As suitable and preferred examples of aromatic diamines that are suitable as component a) of said diamide, mention may be made of: m-, p-, o-xylylenediamine, in particular m- and p-, m-, p-, o-phenylenediamine, in particular m- and p-xylylenediamine.

As examples of monoacids c), mention may be made of: hexanoic, heptanoic, octanoic, nonanoic, decanoic, undecanoic, dodecanoic (or lauric) or stearic acid. The following are preferred: hexanoic, octanoic, nonanoic, decanoic.

The hydroxy acids b) that may replace 12-hydroxystearic acid as already mentioned above are: 9-hydroxystearic and/or 10-hydroxystearic acid, these two acids possibly being above all in the form of a mixture after hydroxylation of oleic acid, which bears an ethylenic unsaturation in position 9, which unsaturation is saturated by the hydroxylation.

Said diamide according to the invention is preferably used in micronized powder form more preferentially with a volume-average size of less than 50 μm and even more preferentially less than 25 μm. More particularly, concerning the distribution, 90% of the particles are smaller than 20 μm. Said size may be determined by laser scattering.

The second subject of the invention concerns an additive, in particular a rheology additive, which comprises at least one diamide (i.e. one or more) as defined according to the invention above and which exists and is used in the form of a paste preactivated and preconcentrated in an organic solvent, preferably a polar organic solvent, and more particularly said additive consists of one or more diamides as defined above according to the invention and of an organic solvent, which is preferably polar, more preferentially with a weight content of said diamide(s) ranging from 15% to 45%.

Another subject of the invention concerns the use of the diamide of the invention as an organogelator in an organic solvent, preferably a polar organic solvent, for producing an organogel, preferably as a rheology agent or additive and more preferentially in a composition preconcentrated in an organic solvent, preferably a polar organic solvent, in the form of a preactivated paste.

The term “polar organic solvent” according to the description above includes in its definition or should be interpreted as meaning “at least one polar organic solvent” or “a mixture of organic solvents comprising at least one polar organic solvent”. A solvent comprising at least one polar group, for instance an alcohol or ester group, is considered as a polar organic solvent. As examples of polar organic solvent according to the first interpretation option, mention may be made of an alcohol such as ethanol or butanol or a mixture thereof, and according to the second interpretation option (mixture of organic solvents comprising at least one polar organic solvent), a mixture of such an alcohol (ethanol or butanol) with a nonpolar solvent, for instance xylene.

More particularly, said use is in coating compositions, in particular paints, varnishes, inks or gel coats or in glue or adhesive, molding, mastic or sealing or stripping or cosmetic compositions.

An organogelator, preferably a rheology agent or additive, which comprises at least one diamide as defined above according to the invention is also covered. More particularly, said organogelator is a rheology agent or additive, in particular a thixotropic agent, in a composition preconcentrated in an organic solvent, preferably a polar organic solvent, in the form of a preactivated paste. The preparation of such preconcentrated preactivated pastes may be performed according to the description and examples of WO 2008/0 153 924, by replacing the described plasticizer with an organic solvent, preferably a polar organic solvent as defined above.

Finally, the invention covers an organic binder composition which comprises, as rheology agent, at least one diamide as defined according to the present invention. More particularly, said organic binder is a binder for coating compositions chosen from paints, varnishes, inks and gel coats or a binder for glue or adhesive compositions or a binder for mastic or sealing or stripping compositions or for molding compositions or, finally, for cosmetic compositions. Preferably, said binder is selected from epoxy resins, vinyl esters, unsaturated and saturated polyesters, alkyds, silanized resins, polyurethanes, polyesteramides, solvent-based acrylic resins with an unreactive solvent, multifunctional acrylate (MFA) monomers and/or oligomers or acrylated acrylic resins with reactive diluent or chlorinated or non-chlorinated elastomers or other chlorinated polymers.

The molding compositions are more particularly molding compositions for composites, in particular reinforced with reinforcing fibers, such as glass or carbon or aramid fibers or for molded parts, such as SMC or BMC or laminated, such as boat hulls or composite boards or parts molded by casting, with application of the composition by spraying with a spray gun or brushing or rollering.

These specific additives make it possible to modify the viscosity of mastic, glue or adhesive compositions, coating compositions such as paints, varnishes, gel coats or inks, or molding or mastic or sealing or stripping or cosmetic compositions.

To be used as an organogelator additive and more particularly a thixotropic additive in a composition for application such as a coating, for instance paint, varnish, gel coat or ink, or a glue or adhesive composition or in a sealing or stripping or mastic composition or a molding or cosmetic composition, said diamide needs to be activated in order to have its thixotropic nature. According to a first preferred option, this may take place independently of the final application composition, in a preconcentrated composition of said diamide in an organic solvent, preferably a polar organic solvent, which is preferably liquid at room temperature and suited to the diamide and to the final application and preactivated in the form of a preactivated paste, as described in the description and in particular in the examples of WO 2008/0 153 924. In this case, this preactivated diamide composition is added to the final application composition without the need for activation insofar as said diamide is added already preactivated in its “preactivated” “preconcentrated” paste composition and adapted for the final application. In this case, the final user, who is the formulator, will have no need to activate his formulation since said preactivated diamide thus added gives this nature as soon as it is mixed into said final application composition, without the need for additional activation and thus allowing improved productivity (reduction of the time for preparation, which thus becomes more practical and simpler).

In the absence of preactivation in the form of a preactivated paste preconcentrated in a medium that is suited to (compatible with) the fmal application, the activation of said diamide may be performed according to a second option, in situ in the final application composition, but by the final user. The diamide of the invention may thus be preactivated in the form of a preactivated and preconcentrated paste.

This activation requires high-speed shear and corresponding heating with temperature increases that may be up to close to 120° C. depending on the products, and also a minimum necessary time, dependent on the temperature conditions and on the system, for developing optimum final rheological properties. These additives give the composition into which they are incorporated thixotropic behavior characterized by pronounced shear-thinning, i.e. a reduction of the viscosity when the shear increases, followed by a time-dependent reacquisition of viscosity (equivalent to a hysteresis effect). Thus, this type of additive gives the final composition excellent application properties which are characterized by a high viscosity at rest, good stability of this viscosity on storage, good anti-sedimentation, ease of application and of extrusion after application and good sagging resistance once applied.

A fatty acid diamide of the invention may be obtained by condensation between at least one primary diamine according to a), a saturated hydroxycarboxylic fatty acid according to b) and optionally in the presence of a monocarboxylic acid according to c), optionally in the presence of a second primary diamine according to d). The reaction product may optionally be diluted in hydrogenated castor oil or optionally in hydroxylated oleic oil and, in this case, at a content ranging from 10% to 100% by weight relative to the total diamide+oil (for example hydrogenated castor oil) and preferably at a content ranging from 20% to 100% by weight. The hydrogenated castor oil may be used for adapting the affinity of the final mixture (diamide+hydrogenated castor oil) relative to the composition of the final application formulation.

In the case of dilution in hydrogenated castor oil, the addition takes place at a temperature of between 140 and 220° C. At the end of the addition, a solid mass is obtained, which is ground in powder form.

Said fatty acid diamide may thus be used in the form of a powder or a preactivated paste as described above.

The examples described below in the experimental section are presented to illustrate the invention and its performance qualities and do not in any way limit the claimed scope.

### EXPERIMENTAL SECTION

### I—Starting Materials Used

TABLE 1
Starting materials used

| | | Commercial | |
|---|---|---|---|
| Product | Function | reference | Supplier |
| Oleic acid | Reagent | Purified oleic acid | VWR |
| Formic acid | Reagent | Formic acid, 96% | Aldrich |
| Perchloric acid | Reagent | Perchloric acid, 70% | Aldrich |
| Sodium hydroxide | Reagent | 6N NaOH | VWR |
| Hydrochloric acid | Reagent | 37% HCl | VWR |
| Hexamethylene- | Diamine | Hexamethylene- | Aldrich |
| diamine | according | diamine 98% | |
| | to a) | | |
| Hexanoic acid | Acid | Hexanoic acid | Aldrich |
| | according | 99% | |
| | to c) | | |
| Ethylenediamine | Diamine | Ethylenedi- | Aldrich |
| | according | amine ≧99.5% (GC) | |
| | to a) | | |
| Epoxy resin | Binder | Araldite ® GZ | Huntsman |
| | | 7071X75 | |
| Epoxy resin | Binder | Araldite ® GY | Huntsman |
| | | 783 BD | |
| Degassing agent | Degassing | Byk ® A530 | Byk |
| | agent | | |
| Dispersant | Dispersant | Disperbyk ® 110 | Byk |
| Titanium dioxide | Pigment | Tiona 595 | Société des |
| | | | Ocres de |
| | | | France |
| Iron oxide | Pigment | Bayferrox ® 915 | Lubrizol |
| Zinc phosphate | Pigment | ZP 10 | HEUCOPHOS |
| Talc | Additive | Finntalc MO5 | Mondo |
| | | | Minerals |
| Silica | Filler | HPF6 | Sibelco |
| n-Butanol | Solvent | n-Butanol | Aldrich |
| Polyamide | Hardener | Crayamid ® 140 | Arkema |
| Xylene | Solvent | Xylene, reagent grade | Aldrich |
| 12-Hydroxystearic | Hydroxy | 12-HSA | Jayant Agro |
| acid | acid | | |
| | according | | |
| | to b) | | |

### II—Methods and Tests Used

The formulations are evaluated with two tests: the sagging resistance test and an evaluation of the viscosity at various speeds.

Sagging Resistance Test

This is performed using a sagging controller (Levelling/Sagging Tester from Sheen Instruments®) which makes it possible to establish the resistance of a coating to sagging due to gravity. This controller, made of stainless steel and having a flat blade, comprises notches of increasing value.

The test consists in depositing various parallel strips of paint of different thicknesses onto a contrast card by means of the sagging controller. The contrast card is immediately placed vertically, the thinnest film at the top. The thickness at which the strips merge indicates the tendency toward sagging.

Evaluation of the Viscosity

This is performed here using a Brookfield® RV machine at 25° C. (spindle: S 4). The speed of the spindle is set at 50 rpm and the viscosity of each paint is measured after its viscosity has stabilized. The operation is repeated for speeds of 20 rpm, 10 rpm, 5 rpm and 1 rpm.

### III—Preparation and Characterization of the Organogelators and Rheology Additives

A) Preparation of a Mixture of 9-Hydroxystearic and 10-Hydroxystearic Acids (9-HSA+10-HSA)

The preparation is based on the hydroxylation of the double bond of a fatty acid (transposed here to oleic acid) as described in Addition of Formic Acid to Olefinic Compounds by H. B. Knights, R. E. Koos and Daniel Swern, May 2, 1953.

Other methods may be used for affording the monohydroxylated fatty acids 9- and 10-HSA.

### Preparation Method Used

319.1 g of oleic acid, 677.6 g of formic acid and 3.3 g of perchloric acid are placed in a 1-liter round-bottomed flask under a nitrogen atmosphere, equipped with a thermometer, Dean-Stark apparatus, a condenser and a stirrer. After 30 minutes at reflux, the excess formic acid is evaporated off under vacuum at 75 mbar and 65° C.

The compound obtained (102 g) is then hydrolyzed with 6N sodium hydroxide solution (100 g). Finally, the product is neutralized by slow addition of fuming hydrochloric acid (64 g) in 66 g of water.

Purification is performed by dissolving the reaction medium in toluene and washing three times successively with 11% NaCl solution. The toluene is then evaporated off and the product is recrystallized from hexane. 27 g of a mixture of monohydroxylated fatty acids 9- and 10-HSA are thus obtained.

B) Preparation of Diamides According to the Invention and Comparative Diamides

### Example 1

### Comparative Diamide A

49.96 g of hexamethylenediamine (i.e. 0.43 mol, 0.86 amine equivalent) and 244.65 g (0.86 mol, 0.86 equivalent) of stearic acid are placed in a 1-liter round-bottomed flask under a nitrogen atmosphere, equipped with a thermometer, Dean-Stark apparatus, a condenser and a stirrer.

The mixture is heated to 200° C. still under a stream of nitrogen. The water eliminated begins to accumulate in the Dean-Stark apparatus at and above 150° C. The reaction is monitored by the acid number and the amine number. When the acid and amine numbers are less than 10 mg KOH/g, the reaction mixture is cooled to 150° C. and then emptied into a silicone-lined mold. Once cooled to room temperature, the product is mechanically micronized by grinding and screening to obtain a fine and controlled granulometry with a mean size obtained of 7 μm.

### Example 2

### Diamide B According to the Invention

49.96 g of hexamethylenediamine (i.e. 0.43 mol, 0.86 amine equivalent) and 260.48 g of a mixture of 9- and 10-hydroxystearic acids (i.e. 0.86 mol, 0.86 acid equivalent) as described above are placed in a 1-liter round-bottomed flask under a nitrogen atmosphere, equipped with a thermometer, Dean-Stark apparatus, a condenser and a stirrer. The mixture is heated to 200° C. still under a stream of nitrogen. The water eliminated accumulates in the Dean-Stark apparatus at and above 150° C. The reaction is monitored by the acid number and the amine number. When the acid and amine numbers are less than 10 mg KOH/g, the reaction mixture is cooled to 150° C. and then emptied into a silicone-lined mold. Once cooled to room temperature, the product is mechanically micronized as in Example 1, with the same mean size.

### Examples 3 and 4

### Comparative Diamide C (3) and Diamide D According to the Invention (4)

The same procedure was used, but with the following reaction components presented in the table below:

TABLE 2
Diamides C and D

| Example | Reagent | Mole | Equivalents |
|---|---|---|---|
| 3 | Ethylenediamine | 0.5 | 1 |
| Comparative | Hexanoic acid | 0.5 | 0.5 |
| Diamide C | Stearic acid | 0.5 | 0.5 |
| 4 | Ethylenediamine | 0.5 | 1 |
| Diamide D | Hexanoic acid | 0.5 | 0.5 |
| according to the | 9- and 10-Hydroxy- | 0.5 | 0.5 |
| invention | stearic acids | | |

### Examples 5 and 6

### Comparative Diamide E (5) and diamide F according to the Invention (6)

The same procedure was used, but with the following reaction components presented in the table below:

TABLE 3
Diamides E and F

| Example | Reagent | Mole | Equivalents |
|---|---|---|---|
| 5 | Ethylenediamine | 0.5 | 1 |
| Comparative | Hexanoic acid | 0.5 | 0.5 |
| Diamide E | 12-Hydroxystearic | 0.5 | 0.5 |
| | acid | | |
| 6 | Ethylenediamine | 0.5 | 1 |
| Diamide F | Hexanoic acid | 0.5 | 0.5 |
| according to the | 9- and 10-Hydroxy- | 0.25 | 0.25 |
| invention | stearic acids | | |
| | 12-Hydroxystearic | 0.25 | 0.25 |
| | acid | | |

C) Organogelling Property for Diamides A and B

20 g of the pre-ground Comparative Example A (volume-average size of 10 μm) and 80 g of xylene are placed in a metal box at room temperature. Using a Dispermat® CV machine equipped with a paddle 4 cm in diameter, the two products are mixed at a speed of 2000 revolutions/minute (or rpm) for 30 minutes at a temperature not exceeding 20° C. by regulating the temperature by circulation of cold water.

Activation: the box is then closed carefully and placed in an oven preheated to 65° C. for 24 hours. Once cooled, and after standing for 4 hours, the mixture remains liquid.

Following the same procedure, Example B according to the invention is, after the activation step and cooling, in the form of a paste that enables a wooden spatula to remain vertical in the paste. This result illustrates perfectly the capacity of the diamide of Example B according to the invention to be an organogelator.

### IV—Evaluation of the Rheological Performance in a Paint Formulation

### Paint Formulations Used for the Evaluation

### 1—Preparation

A formulation known as a millbase is prepared with the proportions given in Table 3, in the following manner:

In a disperser bowl (Dispermill 2075 yellow line, supplier: Erichsen®) heated with a jacket system:

1. Introduction of the epoxy binders and also the dispersant and the degassing agent. Homogenization is performed after 2 minutes at 800 rpm.
2. Introduction of the fillers and pigments followed by grinding at 3000 rpm for 30 minutes using a 7 cm paddle. By means of the jacketed bowl, this step is cooled with a bath of cold water (20° C.).
3. Introduction of solvents.

### 2—Activation

24 hours after the preparation of the millbase (Table 4), the formulation is again dispersed at 3000 rpm using a 4 cm paddle. The diamides C or D are placed in the millbase at a given activation temperature (ranging from 40° C. to 70° C.) for 20 minutes at 3000 rpm.

After adding the diluted hardener (Table 5) to the millbase, the paints are adjusted with a xylene/butanol mixture (1/1) to 0.4 Pa·s (measured on cone 4 at 25° C. at 2500 s-1 with a Brookfield® CAP 1000 machine). The proportions between the hardener and the solvent mixture are defined in Table 4.

After the adjustment, the paint is mixed at 1500 rpm for 2 minutes and then left to stand for 30 minutes.

TABLE 4
Millbase formulation

| Millbase composition | Function | Mass % |
|---|---|---|
| Araldite ® GZ 7071X75 | Binder | 17.3 |
| Araldite ® GY 783 BD | Binder | 12.9 |
| Byk ® A530 | Degassing agent | 0.5 |
| Disperbyk ® 110 | Dispersant | 0.5 |
| Tiona 595 (Titanium dioxide) | Pigment | 1.9 |
| Bayferrox ® 915 595 | Pigment | 4.1 |
| (iron oxide) | | |
| ZP 10 (zinc phosphate) | Pigment | 7.5 |
| Finntalc MO5 | Filler | 9.4 |
| Silica HPF6 | Filler | 19.0 |
| n-Butanol | Solvent | 5.4 |
| Diamide C or D | Rheology | 0.8 |
| | additive | |
| TOTAL | | 79.3 |

TABLE 5
hardener

| Hardener composition | Mass % |
|---|---|
| Crayamid ® 140 | 8.8 |
| Xylene | 11.9 |
| TOTAL | 20.7 |

### 3—Evaluation of the Rheology of the Formulations and Results

Various paint formulations were prepared according to the proportions in Table 3 and 4 with different activation temperatures ranging from 40 to 70° C. according to the protocol mentioned above.

The sagging resistance and rheology results show that diamide D based on 9- and 10-HSA has a thixotropic effect on the formulation once it is activated at 60 or 70° C., unlike diamide C, which is inactive irrespective of the temperature (Table 7). Moreover, diamide D makes it possible to have good sagging resistance (Table 6). Consequently, diamide D based on 9- and 10-HSA has the required characteristics of a rheology additive.

TABLE 6
sagging resistance results

| Test | Diamide | Sagging resistance (μm) |
|---|---|---|
| Example 3, 40° C. | C | 225-250 |
| Example 3, 50° C. | C | 225-250 |
| Example 3, 60° C. | C | 225-250 |
| Example 3, 70° C. | C | 225-250 |
| Example 4, 40° C. | D | 225-250 |
| Example 4, 50° C. | D | 225-250 |
| Example 4, 60° C. | D | 375-400 |
| Example 4, 70° C. | D | 400-425 |

TABLE 7
rheological results

| | | Brookfield viscosity at 25° C. (mPa · s) | | | | |
|---|---|---|---|---|---|---|
| | | | | | 50 | 100 |
| Test | Diamide | 1 rpm | 5 rpm | 10 rpm | rpm | rpm |
| Example 3, 40° C. | C | 2000 | 1150 | 906 | 657 | 588 |
| Example 3, 50° C. | C | 1912 | 1002 | 858 | 642 | 580 |
| Example 3, 60° C. | C | 1970 | 1055 | 880 | 640 | 584 |
| Example 3, 70° C. | C | 1989 | 1107 | 900 | 654 | 588 |
| Example 4, 40° C. | D | 2000 | 1120 | 920 | 660 | 596 |
| Example 4, 50° C. | D | 2400 | 1280 | 1020 | 700 | 630 |
| Example 4, 60° C. | D | 9600 | 3400 | 2300 | 1092 | 862 |
| Example 4, 70° C. | D | 11800 | 4000 | 2640 | 1196 | 922 |

### V—Evaluation of the Rheological Performance of Diamides E (Comparative) and F (According to the Invention) in a Paint Formulation

Paint formulations used for the evaluation: preparation and activation and evaluation, as described for amides C and D in point IV) with the same formulations used and tested, but with activation at only 60° C.

### Results of Evaluation of the Rheology of the Formulations

The sagging resistance and rheology results show that diamide F, based on 9-HSA and 10-HSA and 12-HSA, has a thixotropic effect on the formulation once it is activated at 60° C., like diamide E based on 12-HSA (see Table 9). Moreover, diamide F makes it possible to have good sagging resistance (Table 8). Consequently, diamide E based on 9- and 10-HSA has the necessary and satisfactory characteristics of a rheology additive.

TABLE 8
sagging resistance results for diamides E and F

| Test | Diamide | Sagging resistance (μm) |
|---|---|---|
| Example 5, 60° C. | E | >600 |
| Example 6, 60° C. | F | >600 |

TABLE 9
rheological results

| | | Brookfield viscosity at 25° C. (mPa · s) | | | | |
|---|---|---|---|---|---|---|
| | | | | | 50 | 100 |
| Test | Diamide | 1 rpm | 5 rpm | 10 rpm | rpm | rpm |
| Example 5, 60° C. | E | 22200 | 6760 | 4300 | 1804 | 1308 |
| Example 6, 60° C. | F | 18800 | 8840 | 3720 | 1604 | 1186 |

1. A fatty acid diamide, comprising at least one reaction product of a mixture comprising:
a) at least one diamine selected from the group consisting of:
linear aliphatic C₂ to C₁₂, diamine,
C₆ to C₁₈ cycloaliphatic diamine, and
aromatic diamine,
b) at least one saturated hydroxycarboxylic fatty acid selected from the group consisting of 9-hydroxystearic acid and 10-hydroxystearic acid in the presence or absence of 12-hydroxystearic acid,
c) optionally, at least one monoacid selected from the group consisting of saturated, non-hydroxylated C₆ to C₁₈ linear carboxylic acids,
d) optionally, at least a second diamine different from a) and selected from the group consisting of linear aliphatic C₂ to C₁₂ amines.

2. The fatty acid diamide of claim 1, wherein 12-hydroxystearic acid is present and the percent by weight of 9-hydroxystearic acid and/or 10-hydroxystearic acid is from 10% to 99% by weight of component b).

3. The fatty acid diamide of claim 1, wherein 12-hydroxystearic acid is absent and is replaced 100% with 9-hydroxystearic acid and/or 10-hydroxystearic acid.

4. The diamide as claimed in claim 1 wherein said diamide bears two amide functions based on the same hydroxy acid b) or based on two different hydroxy acids b).

5. The diamide as claimed in claim 1 wherein said monoacid c) is present in an amount such that the b/c mole ratio of said hydroxy acid b) to said monoacid c) is from 1/2 to 4/1.

6. The diamide as claimed in claim 1, said diamide having one amide function based on a hydroxy acid b) and another amide function based on said monoacid c) with a b/c mole ratio of 1/1.

7. The diamide as claimed in claim 1 comprising at least two different reaction product.

8. The diamide of claim 7 comprising a mixture of products, said products having the following formulae:
b1-a1-c1
b1-a1-b1
b1-a1-b2
with a1: diamine residue according to a), b1: 9-hydroxystearic acid residue, b2: 10-hydroxystearic acid residue and c1: non-hydroxylated monoacid residue according to monoacid c).

9. The diamide of claim 1 in the form of micronized powder having a volume-average size of less than 50 μm.

10. A rheology additive comprising at least one diamide according to claim 1 in the form of a paste preactivated and preconcentrated in an organic solvent.

11. (canceled)

12. (canceled)

13. An organogelator comprising at least one diamide according to claim 1.

14. The organogelator of claim 13 which is a rheology agent or additive in a composition preconcentrated in an organic solvent in the form of a preactivated paste.

15. An organic binder composition comprising as rheology agent at least one diamide according to claim 1.

16. The organic binder composition of claim 15 wherein said organic binder is a binder for coating compositions selected from the group consisting of from paints, varnishes, inks and gel coats, or a binder for glue or adhesive compositions or a binder for mastic or sealing or stripping compositions or for molding compositions or for cosmetic compositions.

17. The composition of claim 15 wherein said binder is selected from the group consisting of: epoxy resins, unsaturated and saturated polyesters, vinyl esters, alkyds, silanized resins, polyurethanes, polyesteramides, solvent-based acrylic resins with an unreactive solvent, multifunctional acrylic monomers and oligomers and acrylated acrylic resins with reactive diluent and chlorinated and non-chlorinated elastomers.
